(19) United States
(12) Patent Application Publication
(10) Pub. No.: US 2005/0267484 A1
(43) Pub. Date: Dec. 1, 2005

(54) Extended trochanteric osteotomy guide

(76) Inventor: Jeff Menzner, Boise, ID (US)

Correspondence Address: PEDERSEN & COMPANY, PLLC, P.O. BOX 2666, BOISE, ID, 83701, US

(21) Appl. No.: 11/207,381

(22) Filed: Aug. 18, 2005

(51) Int. Cl. A61B 17/16

(52) U.S. Cl. 606/87

(57) ABSTRACT

A surgical guide for guiding and steady a saw during surgery, especially adapted to perform at least one, and preferably both, an extended trochanteric osteotomy and a trochanteric slide. The guide includes a linear slot and two slots at opposing oblique angles to the linear slot; the slots are adapted to cooperate with a surgical saw. In a preferred version, the guide is reversible so that it may be used on either a left or right leg depending on which leg the surgery is being performed on. The linear slot may be used for cutting the diaphysis of the femur and one oblique slot may be used for cutting a proximal portion of the femur, while the other oblique slot may be used for cutting a distal portion of the femur. The guide may also include a plurality of anchoring holes through which fixation pins may be inserted to secure the guide to the femur while sawing. In the preferred version, the guide may be adapted to cooperate with at least one alignment mechanism for positioning the guide on the diaphysis of the femur in order to determine where the cuts should be made.

### BACKGROUND OF THE INVENTION

1. Field of the Invention

The present invention relates to a surgical guide, and, more particularly, to a surgical guide for performing an extended trochanteric osteotomy.

2. Description of Related Art

The coxal joint or hip joint is the multiaxial ball-and-socket joint between the head of the femur and the acetabulum of the os coxae. The hip joint is secured by a strong fibrous joint capsule, several ligaments, and a number of powerful muscles. The muscles that move the thigh at the hip joint originate from the pelvic girdle and insert at various places along the femur. The anterior muscles that move the thigh at the hip joint are the iliacus and psoas major; they both insert on the lesser trochanter. The posterior muscles that move the thigh at the hip joint include the gluteus maximus (inserts on the gluteal tuberosity of the femur), gluteus medius (inserts on the greater trochanter of the femur), gluteus minimis (inserts on the lateral surface of the greater trochanter), and tensor fasciae latae (inserts on a broad lateral fascia of the thigh called the iliotibial tract). The medial muscles that move the hip joint include the gracilis pectineus, adductor longus, adductor brevis, and adductor magnus. The sciatic nerve passes from the pelvis through the greater sciatic notch of the os coxae and extends down the posterior aspect of the thigh. Many factors, disease processes, or injury, may result in the need for a total hip arthroplasty, which replaces the acetabulum, the head of the femur, or both with a prosthetic component.

Since the introduction of the total hip arthroplasty, wear has been a primary issue in hip arthroplasty. Many of the early total hip arthroplasty components are wearing out, resulting in an increasing number of revision total hip arthroplasties. Although wear continues to remain a problematic issue, the consequences of wear, namely osteolysis and prosthetic loosening, are of even greater concern.

As the components wear, they produce wear particles (the type of wear particles corresponds to the component material) that concentrate in the tissue in periprosthetic areas. The wear particles produce a series of responses on both cellular and tissue levels, such as osteolysis. Osteolysis is bone resorption (breakdown and assimilation of bone through the action of osteoclasts) in response to particulate debris from the wear of arthroplasty components. Osteolysis can occur in a linear pattern and progress along a bone-cement interface or bone-implant interface and contribute to implant loosening. Once stability is lost, the constructed hip joint fails clinically. Osteolysis is often asymptomatic. Patients may present late with implant loosening secondary to osteolysis or even periprosthetic fracture. Furthermore, radiography does not fully depict the degree of osteolysis. Patients often require revision total hip arthroplasty even in the absence of symptoms due to the potential for fracture and the loss of bone, increasing the difficulty of surgery in the long term.

The most frequently used surgical approaches when performing a revision total hip arthroplasty include the direct lateral, anterolateral, and posterior approaches. Commonly used modifications of the most frequently used approaches include the standard trochanteric osteotomy, the trochanteric slide, and the extended proximal femoral osteotomy, also known as an extended trochanteric osteotomy. The posterior approach is used for most simple revisions of loose endoprostheses, short, loose, cemented stems, and straightforward cup revisions.

The standard trochanteric osteotomy is rarely used, due to complications arising from non-union of the trochanter and lateral hip pain due to prominent trochanteric hardware. The trochanteric slide is used when greater exposure of the femoral shaft is necessary to remove implant materials, to treat deformities or fractures, when abductor tension must be adjusted, or to enhance acetabular exposure. The advantage of this technique is the use of the intact vastus lateralis muscle origin to prevent proximal trochanteric migration. The trochanteric slide is generally preferred in primary and revision cases requiring a limited osteotomy. The extended trochanteric osteotomy is recommended for the most difficult femoral revisions. These include the removal of well-fixed cementless and cemented components. It is also useful to assist with difficult cement removal, as well as, difficult primary total hip arthroplasty with deformity of the proximal femur. The extended trochanteric osteotomy removes the trochanter and an anterolateral segment of proximal femur for exposure of the implant-bone interface as well as to allow a straight approach to the distal canal. The insertions of the gluteus medius and minimus, as well as the origin of the vastus lateralis and an anterolateral periosteal and muscle hinge, prevent proximal migration of the trochanter. These soft tissue attachments also ensure an intact blood supply and, combined with the large surface area of the osteotomy, lead to predictable healing. Additionally, with the extended trochanteric osteotomy direct access to the diaphysis is provided, which allows reaming of the diaphysis for revision stem insertion, as well as easy removal of cement through direct visualization of the medullary cavity.

Typically, in an extended trochanteric osteotomy, after the proximal femur has been exposed and mobilized, it is placed in internal rotation so that the posterior aspect of the femur is facing the ceiling. The osteotomy is then marked out along the exposed posterior femur, extending from the quadrate tubercle at the base of the greater trochanter to a predetermined point on the femoral shaft selected to maximally expose the distal cement or interface. The osteotomy is marked just anterolateral to the linea aspera. A high-speed pencil burr is used to make multiple holes along the posterior osteotomy line, extending through the femur to the anterolateral cortex, which is perforated. Following the multiple drill holes, the posterior cut is completed by connecting the holes posteriorly and cutting the cortex laterally at the distal extent of the osteotomy. Osteotomes are used to crack open the anterior cortex and lever the osteotomy anteriorly, creating a controlled fracture through the anterolateral perforations.

During osteotomy, it is difficult if not impossible for the surgeon to create a linear cut when connecting the holes posteriorly; the cut tends to resemble multiple waves rather than a line. This can make it difficult to accurately reattach the extended trochanteric osteotomy. Additionally, it is difficult for a surgeon to perform a trochanteric advancement without reshaping the edges of the bone; if the osteotomy is moved distally to tension the abductors, its edges will no longer match the edges on the femur diaphysis. Further, the non-linear osteotomy tends to result in intraoperative iatrogenic femoral fractures due to stress risers that fracture down the femur or result in the fracturing of the osteotomized segment. A study published by The Journal of Bone & Joint Surgery (Noble, A. R., et al., Mechanical Effects Of The Extended Trochanteric Osteotomy, 87-A(3) The Journal of Bone and Joint Surgery, 521, 521-529 (2005)) showed that the consistent location of the fracture pattern of the specimens tested was “through the transition zone of the distal bevel cut of the extended trochanteric osteotomy. . . . The shape of the distal end of the osteotomy cut may have a significant influence on the overall strength of a femur with an extended trochanteric osteotomy.” These intraoperative fractures can significantly reduce the overall strength of the femur following extended trochanteric osteotomy, and the non-linear edges of the trochanteric osteotomy can make it difficult to reattach the bone and in turn prolong healing after surgery. This process is also time consuming, prolonging operative time. Therefore, the inventor believes there is a need for a device to aid the surgeon in creating a more linear cut in order to reduce the number of intraoperative iatrogenic femoral fractures, aid in reattaching the osteotomy, and expedite the procedure in a more accurate and reproducible fashion. It would also provide a cutting template to advance the trochanter incrementally to retension the hip abductors. Safety, speed and accuracy are all improved with the invented device.

### BRIEF SUMMARY OF THE INVENTION

The present invention relates generally to surgical guides, and, more specifically, to a surgical guide for performing an osteotomy, such as an extended trochanteric osteotomy. The surgical guide is adapted to guide a surgical saw to create at least one linear cut, for example, along the posterolateral proximal femur to prevent intraoperative iatrogenic femoral fractures and create an intact muscle-osseous sleeve.

Preferably, the guide comprises a main body including a linear cutting slot and at least one projection comprising a cutting slot at an oblique angle to the linear cutting slot. The linear slot may be for cutting the diaphysis of the femur, and one oblique slot may be for cutting a distal portion of the femur, and optionally, an additional projection may comprise another opposing oblique slot for cutting a proximal portion of the femur. The oblique slot(s) are preferably oriented in opposing directions relative to each other and the linear slot. The guide may include a plurality of anchoring holes through which fixation pins may be inserted to secure the guide to the femur while sawing. In the preferred embodiment, the guide is fitted with at least one alignment mechanism for positioning the guide on the diaphysis of the femur in order to determine where the cuts should be made. Preferably, the guide is adapted to be reversible in order to use the guide on either a left or right femur depending on which hip is being operated on.

In accordance with the preferred embodiment of the present invention, there is provided a preferred method of using the guide during an extended trochanteric osteotomy. The preferred method of performing an extended trochanteric osteotomy may be generally according to the Younger et al. extended anterolateral proximal trochanteric osteotomy, in which all of the abductors are reflected with the lateral third of the proximal femur. The Younger et al. approach is well understood in the field. Younger, T. I, MD, Bradford, M. S., MD, Magnus, R. E., MD, Paprosky, W. G., MD., Extended Proximal Femoral Osteotomy: A new Technique for femoral revision arthroplasty, 10(3) The Journal of Arthroplasty. 329, 329-338 (1995).

### BRIEF DESCRIPTION OF THE SEVERAL VIEWS OF THE DRAWINGS

FIG. 1 is a perspective view of one embodiment of the invented extended trochanteric osteotomy guide with the lateral side and the left face of the guide shown to best advantage, and wherein the guide is a small sized guide.

FIG. 2 is a plan view of the embodiment shown in FIG. 1 with the left face of the guide shown to best advantage.

FIG. 3 is a view of the embodiment shown in FIGS. 1 and 2 with the top or lateral side (if the guide is placed on the patient) of the guide shown to best advantage.

FIG. 4 is a plan view of one embodiment of an oscillating saw blade.

FIG. 5 is a plan view of one embodiment of an sagittal saw blade.

FIG. 6 is a plan view of one embodiment of a fixation pin.

FIG. 7 is a plan view of an embodiment of a medium sized guide, with the right face of the guide shown to best advantage.

FIG. 8 is a plan view of an embodiment of a large sized guide, with the right face of the guide shown to best advantage.

FIG. 9 is a posterolateral view of a right femur, with the small sized extended trochanteric osteotomy guide of FIGS. 1-3 positioned on the femur, and an oscillating saw 60 blade ready for sawing.

FIG. 10A is a cross-section of the proximal femur with a saw being directed from posterolateral to anterolateral through one embodiment of the guide during a revision total hip arthroplasty, and wherein the femoral component is still in the medullary cavity.

FIG. 10B is a cross-section of the proximal femur as shown in FIG. 10A during an alternative revision total hip arthroplasty, wherein the femoral component has been removed from the medullary cavity prior to sawing, and only the cement remains.

FIG. 10C is a cross-section of the proximal femur as shown in FIGS. 10A and 10B, wherein the guide and saw are being used during a complex primary total hip arthroplasty or in revision arthroplasty with deformity of the proximal femur.

FIG. 11 is a posterolateral view of the femur, illustrating the path of the extended trochanteric osteotomy after using the preferred embodiment of the invented guide.

FIG. 12 is a posterolateral view of the femur, as the osteotomy of FIG. 11 is fully opened by everting the fragment in continuity with overlying muscle.

FIG. 13 is a posterolateral view of the femur after a femoral implant has been inserted and the osteotomy of FIGS. 11 and 12 has been reattached and held in place with multiple cables.

FIG. 14 is a posterolateral view of the femur, illustrating the path and the movement of the extended trochanteric osteotomy after using the preferred embodiment of the invented guide to perform an extended trochanteric osteotomy with advancement and soft tissue tensioning of the abductors.

FIG. 15 is a plan view of an alternative embodiment of a medium size guide, with the right face of the guide shown to best advantage and the non-cutting edges shown as curved.

FIG. 16 is a plan view of an alternative embodiment of a small sized guide, with the right face of the guide shown to best advantage and the non-cutting edges shown as curved.

FIG. 17 is a side perspective view of one embodiment of a removeable alignment mechanism.

FIG. 18 is a top perspective view of the embodiment shown in FIG. 17.

FIG. 19 is a perspective view of one embodiment of a small sized guide wherein a removeable alignment mechanism is inserted in a linear cutting slot in the guide.

### DETAILED DESCRIPTION OF THE INVENTION

Referring to the Figures, there are shown several, but not the only, embodiments of the invented surgical guide for an extended trochanteric osteotomy. In this Description and the Claims, the term “proximal” means toward the center/torso of the body, whereas the term “distal” indicates a point farther from the center/torso of the body. Other directional terms of reference used herein are: “anterior” meaning toward the front; “posterior” meaning toward the back; “superior” meaning toward the head/top; “inferior” meaning away from the head or toward the bottom; “medial” meaning inwardly from the side toward the midline of the body; and, “lateral” meaning outwardly from the midline of the body toward the side; “anterolateral” meaning situated or occurring in front of and to the side; “posterolateral” meaning situated or occurring in the back of and to the side; and “anteroposterior” meaning from the front to the back to the front. All positions and directions refer to the anatomic position of the femur prior to dislocation and rotation of the limb into the operational position.

The preferred embodiment is a surgical guide 100 for performing an extended trochanteric osteotomy. The surgical guide 100 is adapted to guide a surgical saw to create at least one linear cut along the anterolateral proximal femur to prevent intraoperative iatrogenic femoral fractures, create an intact muscle-osseous sleeve, and aid in the healing of the extended trochanteric osteotomy. The guide 100 may be adapted to be reversible to use on either a left or right femur depending on which hip is being operated on. The guide 100 is symmetrical around a center plane C (see FIG. 3) due to the anchoring holes 37 and the slots 35, 40, 45 extending all the way through the guide 100 and the construction of the alignment mechanism 50 also being symmetrical. This symmetry allows the guide 100 to be reversed or “flipped” over this center plane C for use on either a left or right femur. However, movement of the stylus 56 may break this symmetry, but this movement of the stylus 56 permits the guide 100 to be used for either a left or a right femur.

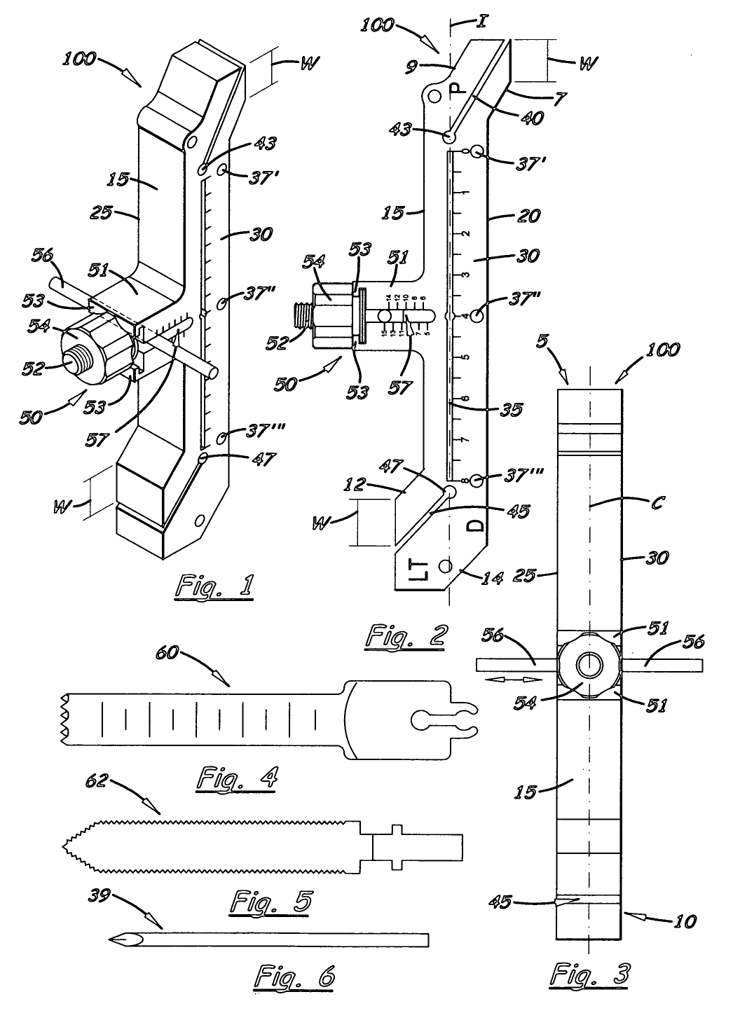

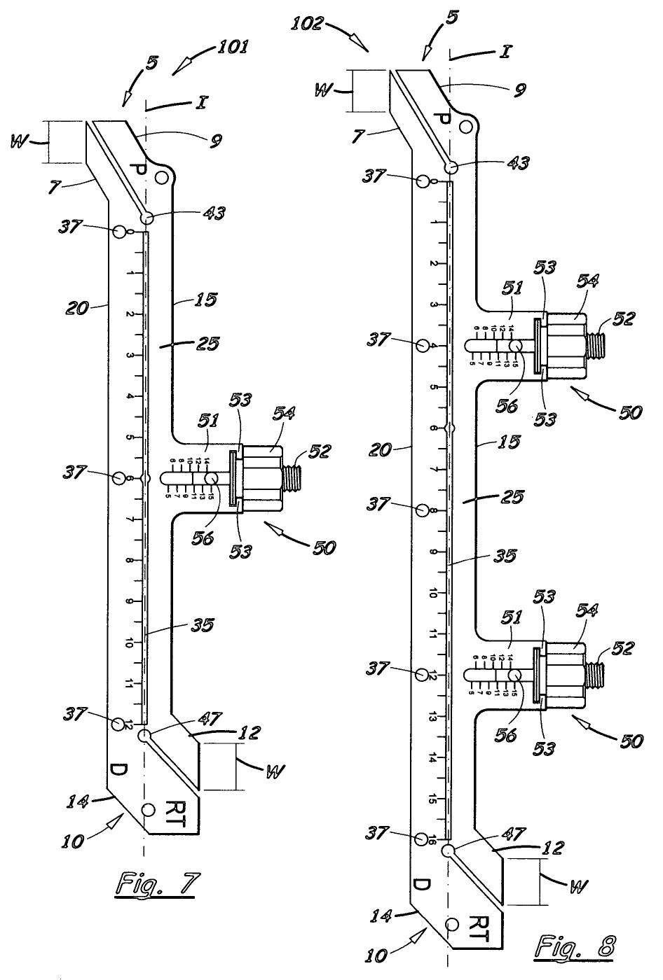

The surgical guide 100 comprises a proximal end 5, a distal end 10, a lateral side 15, a medial side 20, a right face 25, and a left face 30. The proximal 5 and distal 10 ends may be marked with indicia such as “P” for proximal and “D” for distal to help the surgeon orient the guide on the femur; other forms of indica may be used, for example, proximal and distal may be spelled out. Additionally, the right 25 and left 30 faces may also be marked with indicia, such as “L” or “R” for left and right, or alternatively “left” and “right” may be spelled out. Additionally, the guide 100 may comprise measurement markings. The proximal end 5 includes an oblique cutting slot 40, which is preferably linear, a stress relieving hole 43 at one end of the oblique slot 40, a cutting edge 7 and a non-cutting edge 9 (see FIGS. 2, 7, 8, and 15). The distal end 10 includes an oblique cutting slot 45, which is preferably linear, a stress relieving hole 47 at one end of the oblique slot 45, a cutting edge 12, and a non-cutting edge 14 (see FIGS. 2, 7, 8, and 15). A linear slot 35 extends through the main body of the guide 100 from the left face 30 to the right face 25. In the preferred embodiment, a portion of the stress relieving holes 43 and 47 lie on an imaginary line I that extends from the linear slot, as shown in FIGS. 2, 7, 8 and 15. In the preferred embodiment, the linear slot 35 is closed at both ends, and the proximal 40 and distal 45 slots are closed only at one end. The proximal slot 40 is “open” at or near the proximal-most extremity and the distal slot 45 is “open” at or near the distal-most extremity. The planes passing through each of the slots 35, 40, 45 are perpendicular to the right 25 and left 30 faces. The proximal slot 40 is at an angle of 30 degrees in the medial direction from the linear slot 35, and the distal slot 45 is at an angle of 45 degrees in the lateral direction from the linear slot 35; however, the inventor envisions that angles between 20-90 degrees could be used to orient the oblique slots 40, 45 relative to the linear slot 35. This range of angles is preferably between 20-90 degrees because angles closer to 90 degrees lead to stress risers and angles closer to 0 degrees make too long of a cut; however, less preferably, the range may extend from 5-90 degrees.

Preferably, the guide 100 comprises a plurality of anchoring holes 37 that extend through the guide 100 from one face to the other (30 to 25), and the anchoring holes are preferably spaced apart equidistantly. The anchoring holes 37 are adapted to receive fixation pins 39 for securing the guide 100 to the femur 200. The stress relieving holes 43 and 47 may also act as anchoring holes by also receiving fixation pins 39 after being drilled through. These anchoring holes 37 preferably extend through the main body of the guide 100 near the medial side 20. The number of anchoring holes 37 depends on the size of the guide, but at a minimum, the guide 100 should include the two stress relieving holes 43 and 47 and then fixation pins 39 should be inserted into them to ensure stability of the guide 100 on the femur 200.

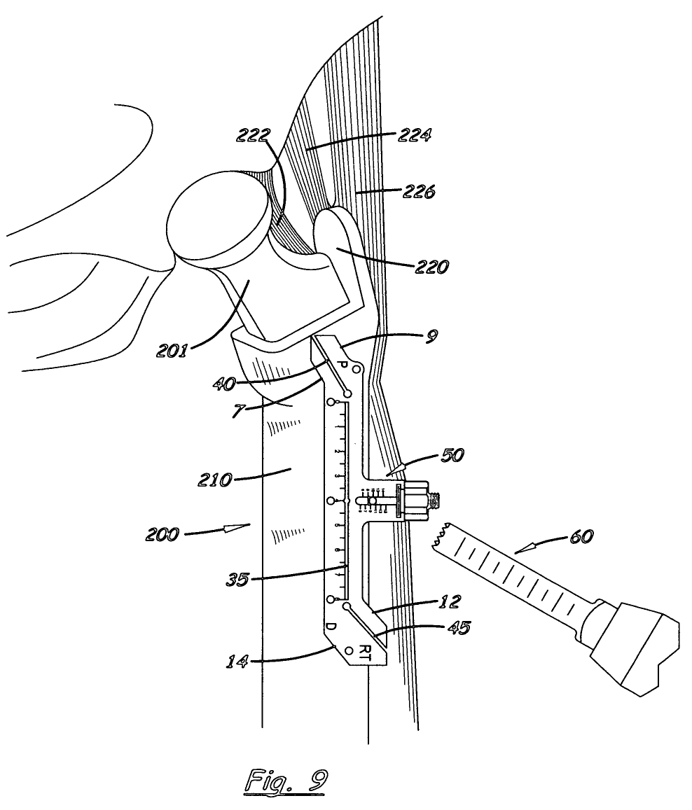

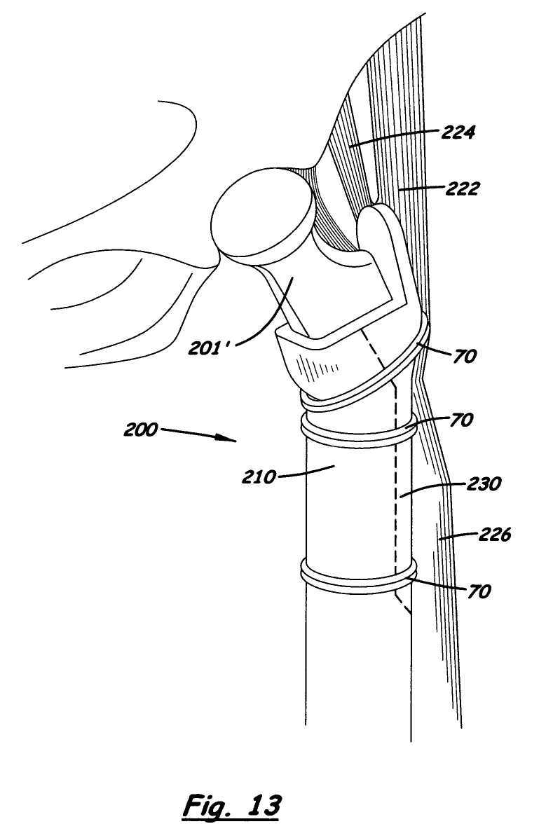

The surgical guide may come in various sizes to accommodate various osteotomy length requirements, such as a small guide 100, a medium guide 101, and a large guide 102 (see FIGS. 1-3, 7 and 8); the small guide 100 is shown in FIGS. 9, 11, 12, 13 for convenience, but the other sized guides may be used as well. The inventor envisions that the preferred dimensions of a small guide 100 will be 8 cm, a medium guide 101 will be 12 cm, and a large guide 102 will be 16 cm (these are the dimensions of the linear slot 35, not the size of the entire guide); however, other sized guides may be manufactured from 4 cm to 25 cm for other osteotomy applications and other lengths for other applications.

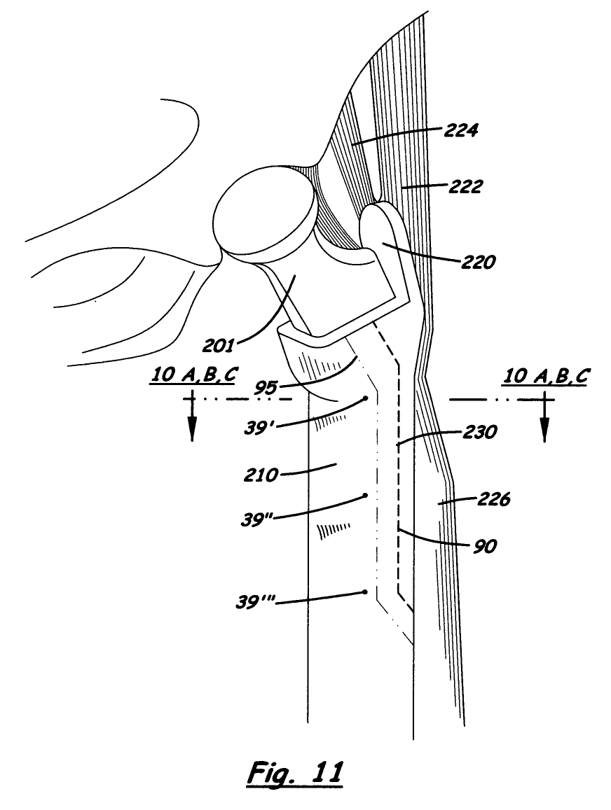

Preferably, the small guide 100 comprises three anchoring holes 37 near its medial side 20, the medium guide 101 comprises four anchoring holes 37, and the large guide 102 comprise five anchoring holes 37 near its medial side 20. The guides 100, 101, 102 may include additional anchoring holes and/or anchoring holes 37 on their proximal 5 and distal 10 ends; however, these anchoring holes 37 are not required, but merely add additional support for holding the guide on the femur 200. The anchoring holes 37 are spaced at an equal distance from each other, preferably 4 cm, but may be anywhere from 1 cm apart to 18 cm apart, in order to allow the surgeon to pick up the guide 100 from the fixation pins 39 (the fixation pins 39 stay in the bone) and move it down a set distance along the femur 200 and then slide the guide back over the fixation pins 39, and still use at least one of the holes created in the femur. For example, if the small guide 100 is being used to perform the extended trochanteric osteotomy and the surgeon needs a longer cut, but does not have a larger guide readily available, the surgeon may move the guide 100 down along the femur 200 by lifting the guide 100 from off the fixation pins 39′, 39″, and 39′″ and then moving the guide down so that fixation pin 39″ was now in anchoring hole 37′, and fixation pin 39′″ was now in anchoring hole 37″ (see FIGS. 2 and 11); another fixation pin 39 could then be inserted into anchoring hole 37′″.

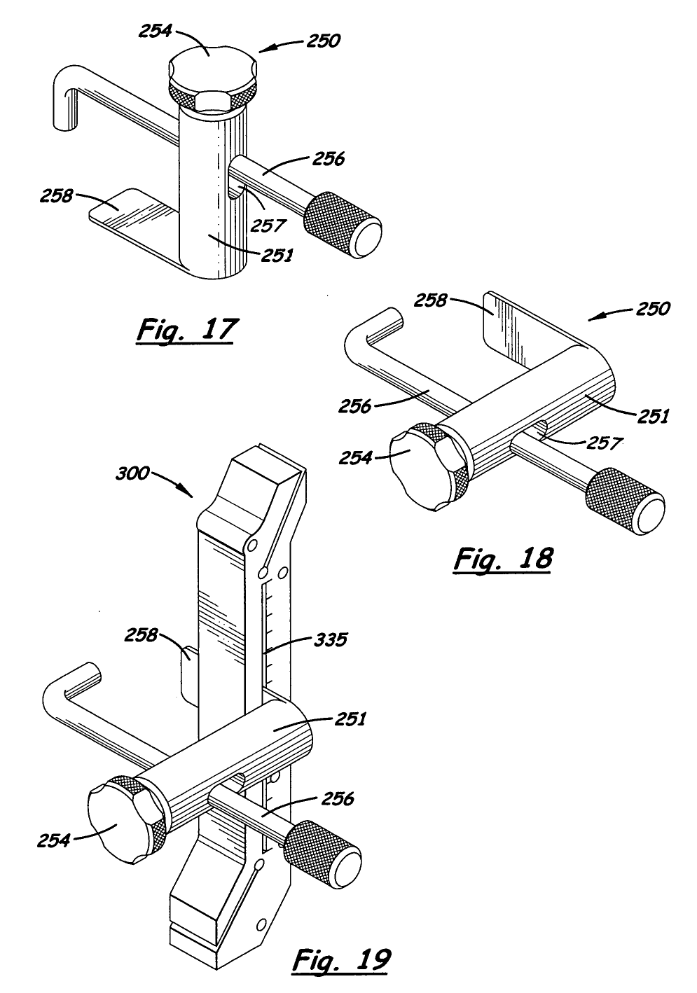

The guide 100 comprises an alignment mechanism 50 for determining where the guide 100 should be placed on the femur 200, in order to determine the depth of the cut. The alignment mechanism 50 may be any of various mechanisms comprising a pin or other stop or elongated member, such as a stylus 56, that is slidably adjustable in a lateral to medial direction toward and away from the linear slot 35 to determine where the guide 100 should be placed on the femur 200 in order to determine the depth of the cut; optionally, the stylus may also be adjustable in a vertical (or in posterior to anterior) direction in order to reverse the guide 100, so it may be placed on either a left or a right femur 200. Preferably, the stylus 56 is generally perpendicular to the longitudinal length of the linear slot 35 and generally parallel to the plane of the slot 35 (i.e. the plane of the linear slot 35 extending into the paper as in FIGS. 2,7 and 8). The alignment mechanism 50 is preferably attached to the lateral side 15 of the guide 100 in order to avoid the sciatic nerve and for ease of use; however, the alignment mechanism 50 may be separate from the guide 100 (see FIGS. 17-19 for an alternative embodiment) and used in combination with the guide 100, such as being inserted into the linear slot 35 of the guide 100. Additionally, one or more alignment mechanisms 50 may be connected to the guide 100, as shown in FIGS. 8, and 16-19 for example.

The alignment mechanism 50 comprises a housing 51 connected to the guide 100, and preferably attached to the lateral side 15 of the guide 100, wherein connected refers to being joined or link together usually by means of something intervening and wherein attached refers to being fixed or fastened to something. The housing 51 includes a bore hole that extends from the interior of the housing 51 through the guide 100 to the medial side 20 of the guide 100. The housing 51 also includes a retaining slot 53 and a stylus slot 57. A base portion of a threaded nut or knob 54 is inserted into the retaining slot 53. A threaded shaft 52 is then inserted into the bore through the medial side 20 of the guide 100 and threadably engages the knob 54. The shaft 52 has an aperture for receiving a stylus 56; the stylus 56 is preferably perpendicular to the shaft 52 and the guide 100. After the stylus 56 is inserted in the shaft 52, the ends of the stylus 56 are crimped, capped, or otherwise manufactured so that the ends of the stylus 56 abut against the edges of the aperture in the shaft 52 allowing the stylus 56 to slidably move back and forth through the aperture without “falling out”. The turning of the knob 54 causes the shaft 52 to rotate, in turn moving the stylus 56 back and forth in the slot 57 toward and away from the linear slot 35.

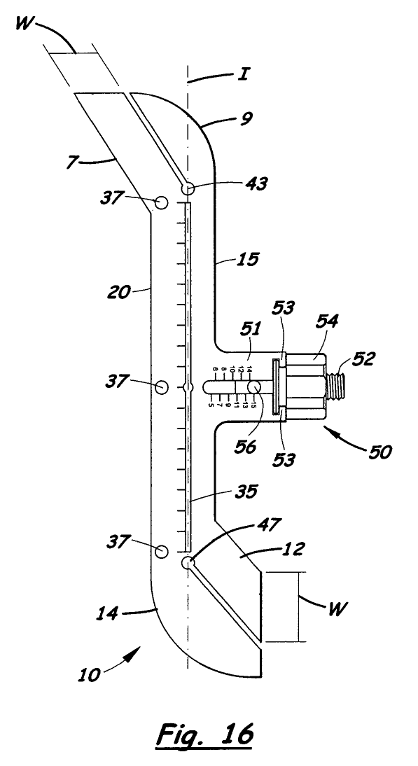

As shown in FIGS. 16-19, an alternative alignment mechanism 250 may be used wherein the alignment mechanism 250 is removeably connected to a guide 300 via a support arm 258. The removeable alignment mechanism 250 comprises a housing 251 having a top portion and a bottom portion, wherein the support arm 258 extends from the bottom portion of the housing 251 perpendicular to the housing 251 and wherein a knob 254 is positioned on the top portion of the housing 251. The housing 251 further comprises a stylus slot 257. The inside of the housing 251 comprises a shaft (not shown) that engages the knob 254. The shaft (not shown) comprises an aperture (not shown) for receiving a stylus 256. The stylus 256 is generally perpendicular to the housing 251 and is generally perpendicular to the longitudinal length of the linear slot 335 and generally parallel to the plane of the slot. The stylus 256 is preferably slidably moveable through the shaft (not shown) in a vertical (or in posterior to anterior) direction in order to reverse the guide 300, so it may be placed on either a left or a right femur 200. Optionally, the stylus 256 may also be adjustable in a lateral to medial direction along the slot 257 toward and away from the linear slot 335 due to the knob 254 rotating the shaft (not shown) in order to determine where the guide 300 should be placed on the femur 200 to determine the depth of the cut. The removeable alignment mechanism 250 may be connected to the guide 200 by inserting the support arm 258 into the linear slot 335. The stylus 256 may then be adjusted to determine the depth of cut to be made. Other styli embodiments or other elongated members may be used so long as they may be adjusted in a lateral to medial direction to determine the depth of cut.

The non-fixed nature or freely slidable nature of the stylus 56 permits the stylus 56 to be used for both the right and left femura due to the tendency of the stylus 56 to “drop down” anteriorly (when the patient is laying prone on an operating table) through the aperture depending on which hip is being operated on, so that the circumference of the stylus 56 is always abutting the lateral surface of the femur no matter which leg is being operated on. Additionally, the stylus 56 is preferably continuously adjustable toward and away from linear slot 35 by means of turning knob 54, wherein the rotation of knob 54 moves the shaft 52 axially toward and away from linear slot 35 carrying the stylus 56 with it. Preferably, the stylus 56 is adjustable at least between 5 mm and 15 mm, so that when the guide is positioned on the femur 200, the osteotomy may be made between 5 mm and 15 mm from the lateral aspect of the femur 200; however, the inventor envisions that wider ranges may be made available, such as between 2 mm and 30 mm, for example. If the osteotomy cut is less than 5 mm, the osteotomy may be too fragile and will break. The inventor approximates 15 mm to be about half way across the diaphysis of a femur, and if a surgeon cuts farther than 15 mm across the diaphysis 210, the surgeon could end up with a small portion of bone on the medial side 20 of the femur 100, which increases the chance for fractures; however, a distance greater or less than 5-15 mm between the lateral aspect of the femur 100 and the femoral component 201 could be cut if necessary. The housing 51 also includes indicia for indicating how far away the femoral component 201 is from the lateral aspect of the femur 200. The indicia preferably range at least from 5 mm to 15 mm.

The preferred method of revision total hip arthroplasty performed in combination with the invented surgical guide is the extended trochanteric osteotomy comprising the Younger et al. approach, with the following preferred adaptations, and with the invented surgical guide being used to ensure the linear cutting of the bone, wide exposure, soft tissue preservation, and trochanteric advancement when needed.

Prior to surgery, a standard lateral radiograph and anteroposterior radiograph is taken of the hip to determine the amount of diaphyseal bone left for component fixation; the level where the metaphyseal flare begins distally, and the relation to the osteotomy; the apex of the anterior bow of the femur; and the distance between the lateral aspect of the femur and the femoral component in order to know where to place the guide so that the saw will pass from the posterolateral aspect of the femur to the anterolateral aspect of the femur, just skirting the edge of the femoral component. The surgeon selects the size of guide, either a small guide 100, medium guide 101, or large guide 102, depending upon the length of the osteotomy required to expose the implant or cement mantle or correct deformity as shown in the radiographs.

In the posterior approach, a standard posterolateral incision is made into the thigh, centered over the tip of the greater trochanter 220 and extending as far distally as is necessary to complete the osteotomy. The short external rotators are incised off the trochanter 220, the proximal insertion of gluteus maximus may be released, and complete posterior, superior, and inferior capsulectomy is performed. Once the proximal femur has been exposed and mobilized, it is placed in internal rotation so that the posterior aspect is facing the ceiling. The vastus lateralis 226 is separated from the femur 200 along its posterior border and held with a Bennett retractor, maintaining its origin on the vastus ridge. The extended trochanteric osteotomy is usually done through a posterolateral approach. It may be done at any time during the procedure—before or after dislocation, or after stem removal. The easiest time is after the stem has been removed; however, this is often not possible, so the osteotomy is done after dislocation, but before stem removal.

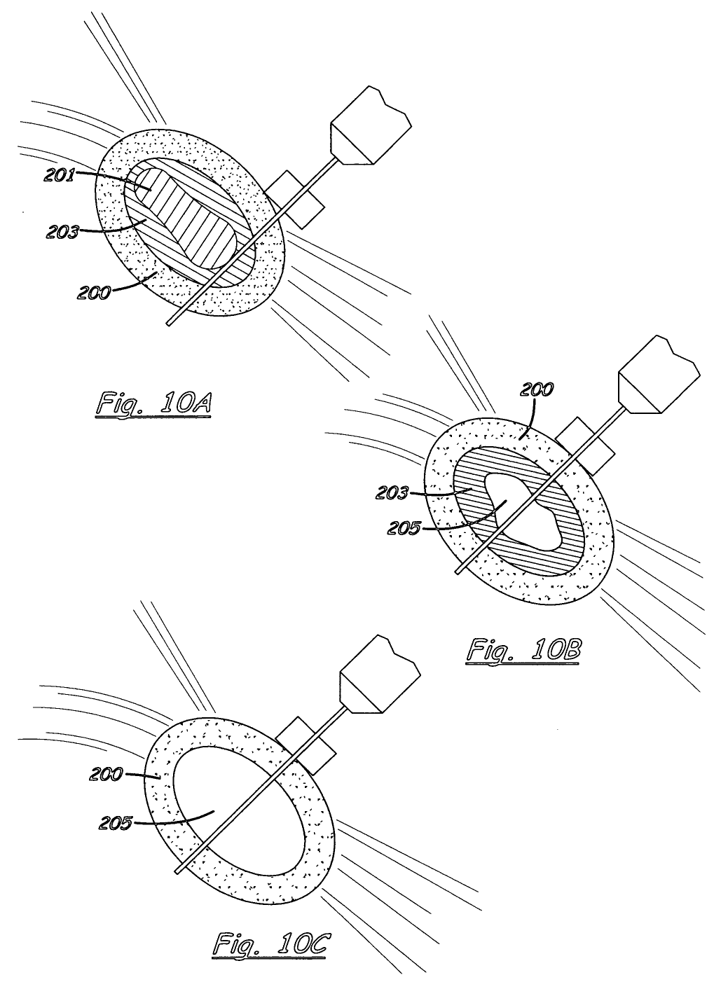

The surgeon then takes the guide (guide 100 is shown, but other sized guides may be used depending on the size needed to perform the osteotomy) and adjusts the alignment mechanism 50 on the guide 100 to the proper distance between the lateral aspect of the patient's femur 200 and the femoral component 201 inside the medullary cavity 205. If the femoral component 201 has already been removed (see FIG. 10B) and only the cement mantle 203 remains in the medullary cavity, or if the surgeon is performing a complex primary total hip arthroplasty in order to fix a severe femoral deformity or remove intraosseous hardware and there is no femoral component 201 inside the femur 200 (see FIG. 10C), the surgeon can decide how much of the diaphyseal circumference he would like to remove (it is not predetermined by the location of a femoral component); usually the surgeon will saw no more than half way across the diaphysis 210, which is approximately 15 mm from the lateral aspect of the femur to half way across the diaphysis 210 (see path 95 illustrated in FIG. 11).

The guide 100 is then oriented so that the proper face (either right 25 or left 30 depending on which femur is being operated on) is showing and the surgeon positions the guide 100 on the femur 200 so that the stylus 56 extends down between the lateral aspect of the femur 200 and the vastus lateralis 226, and abutting against the lateral aspect of the femur 200 (see FIG. 9); the stylus 56 may have to penetrate some muscle in order to extend fully along the lateral aspect of the femur. As shown in FIG. 9, the right face 25 of the guide 100 is showing as the surgeon is operating on the posterolateral aspect of the right femur 200; the left face 30 of the guide 100 lies against the surface of the femur 200. The proximal oblique slot 40 is angled toward the medial aspect of the femur 200 in order to have the cut extend as far away from the greater trochanter 220 as possible to minimize the possibility of fracturing off the greater trochanter 220. The base of the greater trochanter 220 distal to the vastus tubercle is the area most susceptible to fracture. Also, this is the location that is often attenuated by the lateral profile of the previously failed femoral component and subject to osteolysis. The distal oblique slot 45 is angled toward the lateral aspect of the femur 200 in order to cut a transverse portion of the diaphyseal 210 circumference of the femur 200, and to minimize the possibility of a fracture propagating down the diaphysis 210 of the femur 200. The oblique cut made in the proximal oblique slot 40 exits the proximal femur at the base of the previous cut base of the femoral neck.

The guide 100 is positioned on the femur 200 just anterolateral to the linea aspera so that the proximal end 5 is just below the base of the greater trochanter 220 and the distal end 10 is near the apex of the anterior bow of the femur 200. The stylus 56 on the alignment mechanism 50 extends between the lateral aspect of the femur and vastus lateralis 226. While holding the guide 100 on the diaphysis 210 of the femur 200, and with the stylus set at the desired depth of cut, the surgeon drills through the anchoring holes 37 and the stress relieving holes 43, 47. If the femoral component 201 is still in the medullary cavity 205, the drill will extend down only to the surface of the femoral component 201 when drilling through the anchoring holes 37 (approximately 4 mm into the cortex of the femur); if the femoral component 201 is removed, then the drill will extend all the way from the posterolateral cortex to the anterolateral cortex. Regardless of whether the femoral component 201 is still in the medullary cavity 205 or not, the drill will extend all the way from the posterolateral cortex to the anterolateral cortex when drilling through the stress relieving holes 43, 47. The surgeon not only drills through the stress relieving holes 43, 47 in order to insert fixation pins 39, but because the rounded “corners” of these holes, at the ends of the proximal 40 and distal 45 oblique slots, reduce stress risers from forming, which are common with sharp corners. Stress risers can cause fractures to propagate along the femur 200 and/or cause a fracture to break off the greater trochanter 220.

The guide 100 is secured to the femur 200 with a series of fixation pins 39 inserted through anchoring holes 37 and/or stress relieving holes 43, 47 in the guide 10. If the femoral component 201 has been removed, then the fixation pins 39 will extend through both cortices of the femur, which is preferred because the pins are secured at two points instead of just one improving block stability (if the femoral component is still implanted). It is especially important that fixation pins 39 are inserted in the stress relieving holes 43, 47 when the femoral component 201 is still in the medullary cavity 205 so that the fixation pins 39 are securing the guide 100 at both the posterolateral cortex and the anterolateral cortex, not just at the anterolateral cortex. The small guide 100 preferably uses three fixation pins 39; the medium guide 101 preferably uses four fixation pins; and, the large guide 102 preferably uses five fixation pins. However, any number of fixation pins, as few as two, may be used at the surgeon's discretion so long as the guide is securely fixed to the femur to prevent the guide from moving during the osteotomy.

After the guide 100 is secured, the surgeon selects a saw such as an oscillating saw 60 or a sagittal saw 62 (such as those sold by Stryker Instruments) (see FIGS. 4 and 5). The type of saw selected may be decided on the surgeon's own preference, so long as the saw can easily fit in the slots 35, 40, 45 without penetrating too deep or without binding in the slot. The saw (either 60 or 62) is inserted into the linear slot 35 of the guide 100 cutting along the posterolateral osteotomy line, and extending through the medullary cavity 205 of the femur 200 to the anterolateral cortex (see FIGS. 10A-10C). If the femoral component has remained in the medullary canal 205 of the femur 200, the saw will skirt the edge of the femoral component 201 (see FIG. 10A), creating path 90 as illustrated in FIG. 11. If the femoral component has been removed from the medullary canal (see FIGS. 10B and 10C), a path 95 will be made along the femur 200, as shown in FIG. 11. The surgeon then inserts the saw through the distal oblique slot 45 and cuts the cortex laterally at the distal extent of the osteotomy. The surgeon then inserts the saw through the proximal oblique slot 40 and cuts the cortex medially at the proximal extent of the osteotomy. FIG. 11 is a posterolateral view of the femur, illustrating the path of the extended trochanteric osteotomy after the guide 100 has been used and removed.

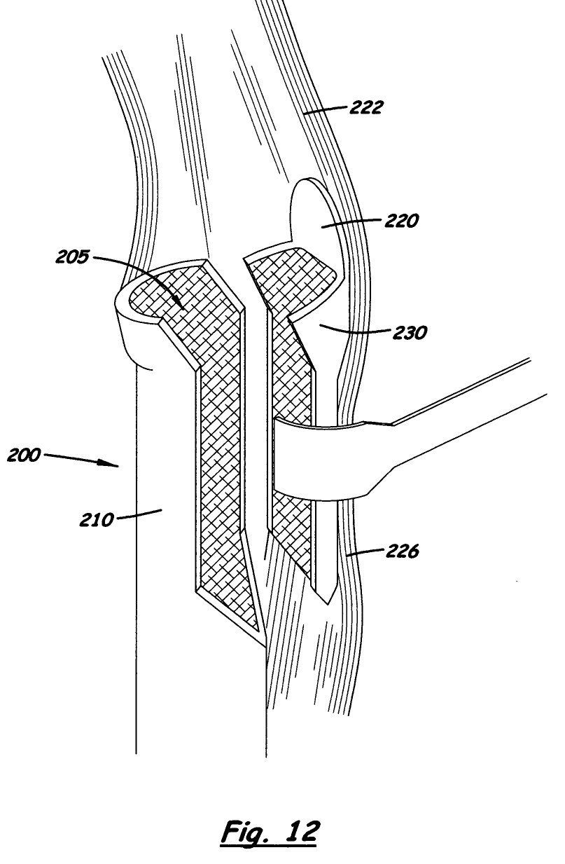

After the cuts have been made, wide osteotomes are used lever the osteotomy or cut portion 230 anteriorly, leaving anterior soft tissue connections undisturbed (see FIG. 12). The gluteus medius 222 and minimus 224 and vastus lateralis 226 remain attached to the cut portion 230 of the femur 200. If the femoral component 201 is still in place, its lateral surface will be completely exposed and there is direct access to its anterior and posterior surfaces. Flexible osteotomes or a high-speed burr may be used to break up the bone-cement or bone-prosthesis interface, or a Gigli saw may be used around the implant to free up the interface. If the femoral component was removed prior to performing the osteotomy, the surgeon can easily access the proximal portion of the medullary canal to remove cement 203. Additionally, if the surgeon is not performing a revision total hip arthroplasty, but is instead performing a primary total hip arthroplasty, this technique provides exposure and access to the proximal femoral diaphysis for removal of hardware, correction of deformity, and safe preparation of the proximal femoral diaphysis, and a large surface area for healing.

Once the revision femoral component 201′ has been inserted, the osteotomy fragment 230 requires very little or no shaping, as it should be straight due to the use of the guide. The osteotomy fragment can be secured prior to final stem preparation or after preparation of the diaphysis for stem insertion. Two or more wires or cables 70 are passed around the diaphysis 210 of the femur 200 and the osteotomy segment 230 to stabilize the osteotomy.

The preferred guide 100 is also designed to facilitate soft tissue tensioning, also called trochanteric advancement, if required. The proximal end 5 comprises a cutting edge 7 and a non-cutting edge 9, and the distal end 10 comprises a cutting edge 12 and a non-cutting edge 14 (see FIGS. 1-3, 7 and 8). The edges 7, 9, 12, and 14 may be surfaces or sides as well, so long as the saw blade is guided along a surface, side, or edge 7, 12 of the proximal and distal ends and prevented from cutting along a surface, side, or edge 9, 14 of the proximal and distal ends. Preferably, the non-cutting edges 9 and 14 are irregularly, or “non-linear” or “non-planar”, shaped, such as jagged, curved, enlarged, or other non-linear or non-planar edges or surfaces relative to the cutting edges 7 and 12 of the distal and proximal ends for example; this will prevent the surgeon from cutting along those edges by providing a physical barrier and a visual reminder (see FIGS. 15 and 16). For example, as illustrated in FIGS. 1-3, 7, and 8, the non-cutting edge 9 of the proximal end 5 of the guide has a protrusion as a deterrent to cutting, and the non-cutting edge 14 of the distal end 10 of the guide is “wider” than the cutting edge 12 of the distal end, visually reminding the surgeon not to cut along that edge. As shown for example in FIG. 15, other means or shapes may be used to deter the surgeon from cutting along those edges, such as protrusions, bumps, recesses, a combination of bumps, protrusions, and/or recesses, enlarged portions, jagged edges, rough texture, curving the edges, color coating, and/or markings. In the preferred embodiment, the cutting edges are the same width W or distance (see FIGS. 1 and 2) to ensure that the same size section is cut out of the bone during soft tissue tensioning of the abductors; preferably, the width is 1 cm.

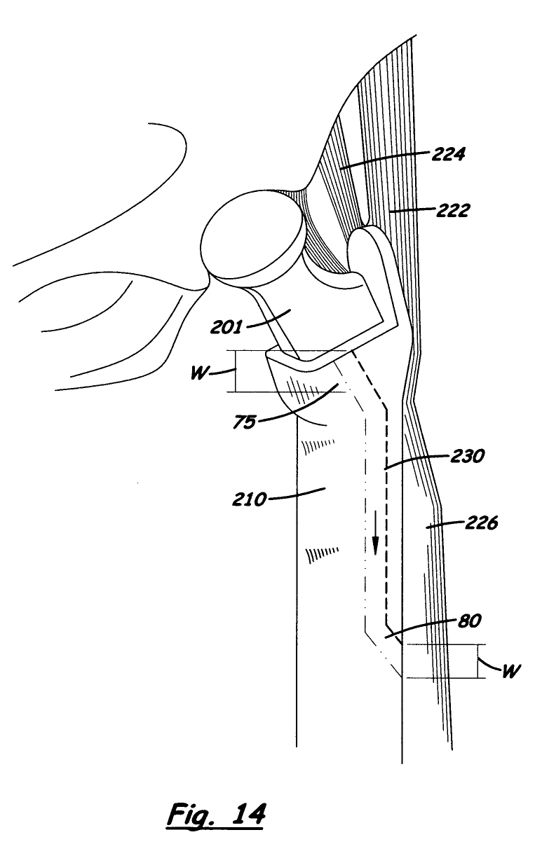

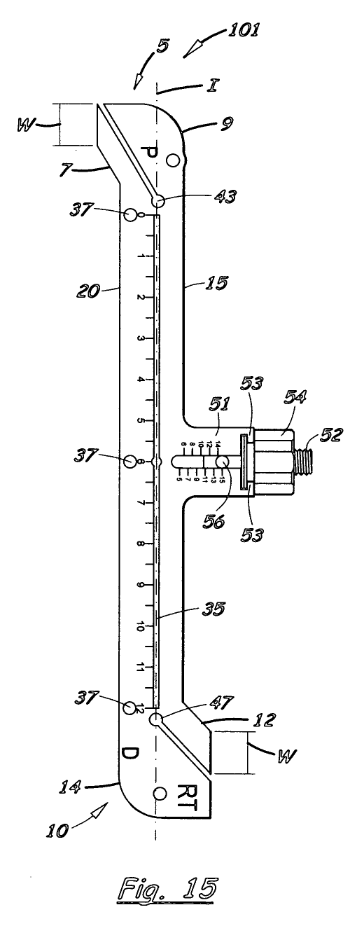

If soft tissue tensioning of the abductors (gluteus medius 222, gluteus minimus 224, and vastus lateralis 226) is required to improve stability of the hip joint, the surgeon can use the cutting edges 7 and 12 to excise proximally and distally and advance the trochanter 220 (attached to the osteotomy 230) distally. After the surgeon has sawed through the linear slot 35, the proximal slot 40, and the distal slot 45, he takes the saw and cuts along the proximal cutting edge 7, excising a portion of bone 75 that is the same width as the distance between the proximal slot 40 and the proximal cutting edge 7 (see FIG. 14). The surgeon then cuts along the distal cutting edge 12, excising another portion of bone 80 that is the same width as the distance between the distal slot 45 and the distal cutting edge 12 (see FIG. 14). These portions of bone are removed, and the trochanter 220 attached to the osteotomy 230 are advanced distally in turn, tensioning the soft tissue.

Preferably, the only securement mechanisms for attaching the device to a bone or other object being cut are pins that pass from one cortex of the femur to the other cortex of the femur, or through only one cortex of the femur. These pins resist proximal and distal movement of the guide relative to the femur and rotation laterally and medially in planes parallel to the posterior surface of the femur. Additionally, one or more styli may extend along the outside surface of the femur in order to set depth of cut from the lateral femoral surface. The guide preferably does not have any clamps that extend substantially around the outside surface of the femur except the stylus, which merely extends along the lateral side of the femur, but does not “wrap around” the femur.

The only structures that protrude or project from the right and left faces of the preferred guide are the pins. Preferably, the pins [defined as a piece of solid material (such as wood or metal) used especially for fastening separate articles together or as a support by which on article may be suspended from another] are elongated, thin, and substantially a single diameter all along the length except for the preferred crimping or capping of the stylus ends. Preferably, the pins and stylus have no pads or enlarged structures at their ends greater than ⅛″, and preferably no crimping or enlarged structure extends more than 5% down the length of the pin or stylus. Preferably, the pins and stylus are not threaded, and are not screws or bolts.

In an alternative, less preferred embodiment, the guide may not include the two oblique slots, but merely be a single elongated slot with (or without) stress relieving holes at each end. The surgeon could then free-hand (meaning the surgeon would not use any means of guiding the saw except for his own hands) the proximal and distal cuts along the femur without using the guide at all, and instead estimating where the cuts should go, or alternatively, the oblique slots may be removed from the proximal and distal ends and the surgeon could cut along an “outer” edge, plane, or portion of the proximal and distal ends. This is a less preferred embodiment because it is highly likely that the proximal and distal cuts will not be straight, making it more difficult to reattach the osteotomy, and increasing the chance for stress risers. Additionally, with the saw blade not being “captured” in the oblique slots, there is an increased risk that the saw will cut in an undesirable direction.

Further, in an additional, alternative, less preferred embodiment, the guide may include a single elongated slot and only one oblique slot; preferably, the oblique slot is the distal oblique slot. The surgeon could then either free-hand the proximal cut, or the elongated slot could extend all the way to the proximal-most portion of the femur. This is also a less preferred embodiment, because there is a greater risk of stress risers due to the possibility of a non-linear proximal cut, which may result in the breaking of the trochanter.

Therefore, the guide may be described broadly as comprising a main body having an upper surface and a lower surface, a distal end, and a proximal end. The guide preferably has an elongated first slot through the main body from the upper surface to the lower surface defining a first vertical plane, and a second slot through said distal end defining a second vertical plane, wherein said second vertical plane is at an oblique angle to said first plane. The saw guide may further comprise a third slot through the proximal end defining a third vertical plane, wherein said third vertical plane is at an opposing oblique angle to said first plane. Additionally, the saw guide may comprise at least one alignment mechanism having an elongated member protruding vertically from said alignment mechanism and moveable toward and away from the first plane.

Although this invention has been described above with reference to particular means, materials and embodiments, it is to be understood that the invention is not limited to these disclosed particulars, but extends instead to all equivalents within the scope of the following claims.

1. A saw guide for performing an osteotomy, the saw guide comprising:
a main body having an upper surface and a lower surface, a distal end and a proximal end;
an elongated first slot through the main body from the upper surface to the lower surface and defining a first vertical plane;
a second slot through said distal end defining a second vertical plane, wherein said second vertical plane is at an oblique angle to said first plane.

2. A saw guide as in claim 1, further comprising a third slot through said proximal end defining a third vertical plane, wherein said third vertical plane is at an opposing oblique angle to said first plane.

3. A saw guide as in claim 1, wherein the guide comprises at least one alignment mechanism comprising an elongated member protruding vertically from said alignment mechanism and moveable toward and away from the first plane.

4. A saw guide comprising:
a main body including a right face and an opposing left face, and a lateral side and an opposing medial side;
a proximal end comprising a proximal cutting slot extending through said proximal end;
a distal end comprising a distal cutting slot extending through said distal end;
a linear cutting slot extending through said main body;
two or more holes extending through said main body; and
wherein said linear cutting slot is closed at both ends, and wherein said proximal and distal cutting slots each comprise one closed end and one open end.

5. A saw guide as in claim 4, wherein said proximal cutting slot and said distal cutting slot are at opposing oblique angles relative to the linear cutting slot.

6. A saw guide as in claim 4, wherein said proximal, distal, and linear cutting slots each define a plane, and wherein each of said planes is perpendicular to said right and left faces.

7. A saw guide as in claim 5, wherein said oblique angles are between 20 and 90 degrees relative to said linear cutting slot.

8. A saw guide as in claim 4, wherein one of said holes is a stress relieving hole at said closed end of the proximal slot, and wherein another of said holes is a stress relieving hole at said closed end of the distal slot.

9. A saw guide as in claim 4, wherein said guide is reversible between said right and left faces.

10. A saw guide as in claim 4, wherein said guide comprises at least one alignment mechanism.

11. A saw guide as in claim 10, wherein said alignment mechanism comprises a stylus, and wherein the stylus is continuously adjustable toward and away from the linear slot by means of turning a knob, wherein the rotation of knob moves a shaft axially toward and away from the linear slot carrying the stylus with it.

12. A saw guide as in claim 11, wherein said stylus is adjustable toward and away from the linear slot at least a distance between 5 mm and 15 mm.

13. A saw guide as in claim 10, wherein the alignment mechanism comprises a housing configured to receive a shaft which engages a knob, and wherein said shaft receives a stylus that is slidably moveable through the shaft and along a slot in the housing due to the knob rotating the shaft.

14. A saw guide as in claim 12, wherein said stylus is perpendicular to the shaft and a longitudinal length of the linear cutting slot.

15. A surgical saw guide configured to be placed on a proximal posterolateral portion of a femur during a trochanteric osteotomy comprising:
an elongated body having a right face and a left face and a lateral side and a medial side;
a proximal end and a distal end;
a linear cutting slot extending through said elongated body from the left face to the right face, wherein said linear slot is closed at both ends;
at least two stress relieving holes through said left face to said right face, wherein a portion of said stress relieving holes lie on points along an imaginary line extending from the linear cutting slot;
an alignment mechanism connected to the guide and configured to align said guide with a lateral aspect of the femur and an interior portion of a medullary cavity.

16. A surgical saw guide as in claim 15, wherein said proximal end includes a proximal cutting slot comprising a closed end and an open end, wherein said closed end forms one of said two stress relieving holes, and wherein said distal end includes a distal cutting slot comprising a closed end and an open end, wherein said closed end forms another of said two stress relieving holes.

17. A surgical saw guide as in claim 15, wherein said saw guide is reversible between the left face and the right face.

18. A surgical saw guide as in claim 15, wherein said alignment mechanism comprises a stylus extending perpendicularly from the guide and the alignment mechanism, and wherein said stylus may be slidably adjusted through the alignment mechanism and retained by the alignment mechanism.

19. A surgical saw guide as in claim 15, wherein said alignment mechanism is removeably connected to the guide via a support arm being inserted into the linear cutting slot, and comprises a stylus extending perpendicularly from the guide and the alignment mechanism, and wherein said stylus may be slidably adjusted through the alignment mechanism and retained by the alignment mechanism.

20. A surgical saw guide as in claim 18, wherein the stylus is continuously adjustable toward and away from the linear slot at least a distance between 5 mm and 15 mm by means of turning a knob, wherein the rotation of knob moves a shaft axially toward and away from the linear slot carrying the stylus with it.

21. A surgical saw guide as in claim 16, wherein the proximal and distal ends each comprise a cutting edge and a non-cutting edge.

22. A surgical saw guide as in claim 21, wherein the non-cutting edge comprises a shape selected from the group consisting of a protrusion, a recess, a combination of protrusions and recesses, an enlarged portion, a curve or multiple curves, and jagged edges.

23. A surgical saw guide as in claim 21, wherein there is a first distance between the cutting edge of the proximal end and the proximal cutting slot and a second distance between the cutting edge of the distal end and the distal cutting slot, and wherein the first distance and the second distance are the same.

24. A surgical saw guide as in claim 15, further comprising: anchoring holes; and pins secured in said anchoring holes and said stress relieving holes.

25. A surgical saw guide as in claim 24, wherein the only structures that project from the right and left faces of the guide are the pins.

26. A surgical saw guide as in claim 15, wherein: the guide does not comprise any clamp or other device that is adapted to wrap around an outside surface of the femur; and wherein the guide does not comprise any securement mechanism that is adapted to contact a medial surface of the femur.

27. A saw guide configured to be placed on a proximal posterolateral portion of a femur and receive a surgical saw during an extended trochanteric osteotomy, the saw guide comprising:
a main body having a lateral side, a medial side, a right face, a left face, a linear cutting slot extending from the left face to the right face configured to receive a surgical saw to cut the diaphysis of the femur, and a plurality of holes for receiving fixation pins that hold the guide on the femur;
two projections at opposing oblique angles to the main body of the guide, wherein a first projection of said two projections is configured to be placed near a proximal portion of the femur and wherein a second projection of said two projections is configured to be placed near a distal portion of the femur, and wherein the first projection comprises a first cutting slot configured to receive a surgical saw, and wherein the second projection comprises a second cutting slot configured to receive a surgical saw;
one or more alignment mechanisms adapted to position the guide on the diaphysis of the femur; and
wherein said guide and alignment mechanisms are reversible between the right and left faces in order to be used on a left or right femur.

28. A saw guide as in claim 27, wherein the main body of the guide, the two projections, and the alignment mechanism are marked with indicia.
